(19) United States
(12) Patent Application Publication
(10) Pub. No.: US 2026/0327679 A1
(43) Pub. Date: Oct. 22, 2026

(54) MAGNETIC BEAD RECOVERY METHOD AND MAGNETIC BEAD RECOVERY DEVICE

(71) Applicant: KAWASAKI INTTITUTE OF INDUSTRIAL PROMOTION, Kawasaki-shi, Kanagawa (JP)

(72) Inventors: Shingo UENO, Kawasaki-shi (JP); Shoichi TSUCHIYA, Kawasaki-shi (JP)

(73) Assignee: KAWASAKI INTTITUTE OF INDUSTRIAL PROMOTION, Kawasaki-shi, Kanagawa (JP)

(21) Appl. No.: 19/105,919

(22) PCT Filed: Aug. 30, 2023
(86) PCT No.: PCT/JP2023/031569

§ 371 Date: Feb. 24, 2025

(30) Foreign Application Priority Data
Aug. 31, 2022 (JP) 2022-138151

(51) Int. Cl. C12Q 1/6806; B03C 1/01; B03C 1/28

(52) U.S. Cl. CPC C12Q 1/6806; B03C 1/01; B03C 1/288; B03C 2201/18; B03C 2201/26

(57) ABSTRACT

In a magnetic bead recovery method, a magnetic bead (1) is recovered from a storage section (11) containing the magnetic bead (1) and a first hydrophilic liquid (2). The magnetic bead recovery method comprises: a step for bringing a capillary (5) filled with a second hydrophilic liquid (302) in a range including a tip (5a) close to the storage section (11) through a hydrophilic liquid layer (121) and coalescing the second hydrophilic liquid (302) with the first hydrophilic liquid (2) in the storage section (11); and a step for introducing the magnetic bead (1) by a magnetic force into the capillary (5) through the first hydrophilic liquid (2) and the second hydrophilic liquid (302).

### TECHNICAL FIELD

The present invention relates to a magnetic bead recovery method and a magnetic bead recovery device.

Priority is claimed on Japanese Patent Application No. 2022-138151, filed on Aug. 31, 2022, the contents of which is incorporated herein by reference.

### BACKGROUND ART

There is a method for immobilizing a biomolecule on a bead for the purpose of observing and detecting the function of a biomolecule. The following method is useful for analyzing a biomolecule: placing bead with immobilized biomolecule into a closed space, such as an emulsion. The reaction of the biomolecule on the bead is conducted within the closed space. The bead is then recovered from the closed space, and the biomolecule on the bead is analyzed (see Non-Patent Literature 1, for example).

On the other hand, there is a technology that uses micro-well array sealed with oil as a closed reaction space containing bead. According to this technology, parallel measurements become possible, improving analysis efficiency (see Non-Patent Literature 2, for example).

To analyze the function of a functional protein, the following method can be used: Distribute bead with immobilized protein and DNA into microwell (housing chambers). Seal the microwell with oil. Analyze the function of the protein within the microwell (see Patent Literature 1, for example).

### CITATION LIST

### Non-patent Literature

Non-Patent Literature 1: K. Matula et al., Single-cell analysis using droplet microfluidics. Advanced Biosystems, 4(1) (2020) 1900188.

Non-Patent Literature 1: J. Yuan et al., An automated microwell platform for large-scale single cell RNA-Seq. Sci. Rep. 6, 33883(2016 ).

### Patent Literature

Patent Literature 1: WO2020/095985

### SUMMARY OF INVENTION

### Technical Problem

In the above-mentioned technology, to analyze the DNA on the bead in order to identify the gene of high-functional protein, it is necessary to recover the bead from the oil-sealed microwell (housing chamber). However, the operation of recovering the bead from the oil-sealed microwell was not easy. Therefore, there was a need for a method to easily recover the bead.

One aspect of the present invention aims to provide a magnetic bead recovery method and a magnetic bead recovery device that can easily recover the bead from the housing chamber.

### Solution to Problem

The present invention includes the following aspects:
1. A method for recovering a magnetic bead from a housing chamber containing the magnetic bead and a first hydrophilic liquid, covered by a hydrophobic liquid layer, comprising: a step of bringing a capillary, filled with a second hydrophilic liquid up to its tip, close to the housing chamber through the hydrophobic liquid layer, and merging the second hydrophilic liquid with the first hydrophilic liquid in the housing chamber; and a step of guiding the magnetic bead into the capillary through the first and second hydrophilic liquids by magnetic force.
2. The method for recovering the magnetic bead according to [1], wherein the second hydrophilic liquid is protruded from the tip when bringing the capillary close to the housing chamber.
3. The method for recovering the magnetic bead according to [1], wherein the housing chamber is a recess formed on one surface of the substrate.
4. The method for recovering the magnetic bead according to any one of [1] to [3], further comprising a step of discharging the magnetic bead from the capillary.
5. A device for a magnetic bead recovery comprising: a capillary positioned opposite a housing chamber containing a magnetic bead and a first hydrophilic liquid; a magnet applying a magnetic attraction force to the magnetic bead; a movement mechanism for moving the capillary toward and away from the housing chamber; wherein a hydrophobic liquid layer covering the first hydrophilic liquid is formed in the housing chamber; and the movement mechanism brings the capillary, filled with a second hydrophilic liquid up to its tip, close to the housing chamber through the hydrophobic liquid layer.

### Effects of Invention

According to one aspect of the present invention, it is possible to provide a magnetic bead recovery method and a magnetic bead recovery device that can easily recover the bead from the housing chamber.

### BRIEF DESCRIPTION OF DRAWINGS

FIG. 1 The configuration diagram of a magnetic bead recovery device according to an embodiment.

FIG. 2 The process diagram of the magnetic bead recovery method according to an embodiment.

FIG. 3 The process diagram of the magnetic bead recovery method according to an embodiment.

FIG. 4 The process diagram of the magnetic bead recovery method according to an embodiment.

FIG. 5 The process diagram of the magnetic bead recovery method according to an embodiment.

FIG. 6 The explanatory diagram of the magnetic bead recovery method according to the first comparative example.

FIG. 7 The explanatory diagram of the magnetic bead recovery method according to the second comparative example.

FIG. 8 (A) The microscopic image of the microwells before magnetic bead recovery. (B) The microscopic image of the microwells after magnetic bead recovery.

FIG. 9 (A) The microscopic image of the magnetic bead and the glass capillary before discharge. (B) The microscopic image of the magnetic bead and the glass capillary after discharge.

### DESCRIPTION OF EMBODIMENTS

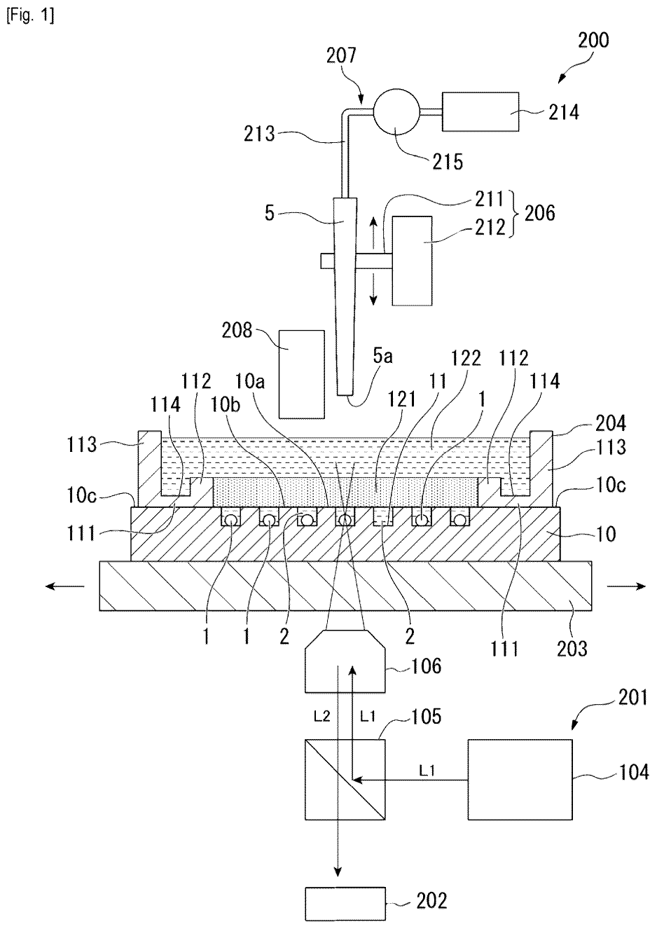

FIG. 1 is the configuration diagram of a magnetic bead recovery device 200 according to an embodiment. Hereinafter, the vertical positional relationship is temporarily defined based on FIG. 1. AN array substrate 10 is positioned with a main surface 10a facing upward.

### Substrate

As shown in FIG. 1, the array substrate 10 has multiple housing chambers (microwells) 11 formed on its main surface 10a, which is one of its surfaces. The array substrate 10 is, for example, rectangular in plan view. The array substrate 10 is flat, and the main surface 10a is planar. The array substrate 10 is an example of a “substrate.”

It is preferred that the array substrate 10 is transparent. When the array substrate 10 is transparent, it is easier to observe the sample in the housing chamber 11. The light transmittance of the array substrate 10 is not particularly limited.

The housing chamber 11 is a recess formed on the main surface 10a. The housing chamber 11 can house sample. Since the array substrate 10 has a plurality of housing chamber 11, it has the advantage of being able to process multiple samples at once.

The internal dimension (e.g., internal diameter) and depth of the housing chamber 11 are determined according to the size of the magnetic bead 1 to be housed. It is desirable that the internal dimensions and depth of the housing chamber 11 be slightly larger than the average particle diameter of the magnetic bead 1. The internal dimension and depth of the housing chamber 11 may be, for example, greater than one time and less than two times the average particle diameter of the magnetic bead 1. This makes it easier for the housing chamber 11 to house only one magnetic bead 1.

The shape of the housing chamber 11 in plan view is, for example, circular. The housing chamber 11 has, for example, a cylindrical internal space.

The shape of the housing chamber 11 is not particularly limited. For example, the housing chamber 11 may have a shape in which the internal diameter decreases in the depth direction. The plan view shape of the housing chamber 11 may also be polygonal, such as rectangular or hexagonal.

A plurality of housing chamber 11 is, for example, arranged in a two-dimensional matrix in multiple rows and columns.

The material for the array substrate 10 includes resin, glass, quartz, and the like. Example of resin includes cycloolefin polymer (COP), polystyrene (PS), polycarbonate (PC), cyclic olefin copolymer (COC), polydimethylsiloxane (PDMS), polyethylene terephthalate (PET), acrylic resin (such as PMMA), and the like. The array substrate 10 may be a laminated structure comprising a flat base material and a well structure formed on the base material. The well structure (housing chamber) is formed in the well structure. The base material is made of glass, quartz, silicon wafer, and the like. The well structure is formed of photoresist, and the like.

It is desirable that the inner surface of the housing chamber 11 be hydrophilized. Hydrophilization treatment method includes oxygen plasma irradiation and ultraviolet-ozone treatment. When the inner surface of the housing chamber 11 is hydrophilized, it is easy to introduce the first hydrophilic liquid 2 into the housing chamber 11.

It is desirable for the main surface 10a to be hydrophobized. Treatment agent for hydrophobizing the main surface 10a of the array substrate 10 includes hexamethyldisilazane, organochlorosilane, and polyorganosiloxane and the like. For example, an array substrate 10 having a hydrophobized main surface 10a can be obtained by pressing a polypropylene film vapor-deposited with hexamethyldisilazane (HMDS) onto the main surface 10a to transfer HMDS to the main surface 10a.

The main surface 10a has a central region 10b and a peripheral region 10c. The housing chamber 11 is formed in the central region 10b. The peripheral region 10c surrounds the central region 10b. The housing chamber 11 houses the magnetic bead 1 and the first hydrophilic liquid 2. The magnetic bead 1 is, for example, immobilized with a biomolecule such as nucleic acid, peptide, and protein as target. The first hydrophilic liquid 2 is, for example, water or an aqueous solution. The magnetic bead 1 is particle containing a magnetic material. Example of a magnetic material includes iron, nickel, cobalt, or their oxides. The average particle diameter of the magnetic bead 1 may be, for example, 1 μm to 10 μm. The average particle diameter of the magnetic bead 1 is preferably 1 μm to 5 μm, and more preferably 2 μm to 4 μm.

### Magnetic Bead Recovery Device

The magnetic bead recovery device according to the embodiment will be described.

The magnetic bead recovery device 200 includes an optical system 201, a photodetector 202, a stage 203, a water-stacking frame (liquid stacking frame) 204, a capillary 5, a height adjustment mechanism (moving mechanism) 206, a liquid supply system 207, and a magnet 208.

The optical system 201 includes a light source 104, a half mirror 105, and an objective lens 106.

The light source 104 can be a mercury lamp, halogen lamp, laser light source, xenon lamp, or LED. The wavelength of the emitted light L1 from the light source 104 is not particularly limited, but may include, for example, the visible light region (wavelength 360 nm to 780 nm).

The half mirror 105 reflects the emitted light L1 and directs it towards the array substrate 10. The half mirror 105 can irradiate the array substrate 10 with the emitted light L1 from the lower side (opposite to the capillary 5) relative to the array substrate 10.

The objective lens 106 is installed on the lower side (opposite to the capillary 5) relative to the array substrate 10.

The optical system 201 irradiates the array substrate 10 on the stage 203 with the emitted light L1 from the light source 104 through the half mirror 105 and the objective lens 106. The reflected light L2, which is reflected within the housing chamber 11, passes through the half mirror 105 and reaches the photodetector 202.

The photodetector 202 includes, for example, an imaging device such as a CCD (Charge-Coupled Device) or CMOS (Complementary Metal-Oxide-Semiconductor).

The stage 203 is where the array substrate 10 is placed. The stage 203 is movable in any direction within the horizontal plane.

The water-stacking frame 204 includes a bottom wall 111, an inner peripheral wall 112, and an outer peripheral wall 113. The bottom wall 111 is annular. The inner peripheral wall 112 is erected on the inner peripheral edge of the bottom wall 111. The outer peripheral wall 113 is erected on the outer peripheral edge of the bottom wall 111. An annular groove 114 is formed between the inner peripheral wall 112 and the outer peripheral wall 113. The water-stacking frame 204 is provided on the main surface 10a of the array substrate 10. The water-stacking frame 204 surrounds all the housing chamber 11 in plan view.

The capillary 5 is a tubular body formed of a material such as glass or resin. The capillary 5 is, for example, cylindrical. The capillary 5 is positioned with its tip 5a facing downward. The tip 5a faces the main surface 10a of the array substrate 10. The capillary 5 is filled with a hydrophilic liquid (e.g., water or an aqueous solution). The capillary 5 is provided above the array substrate 10.

The internal diameter of the tip 5a of the capillary 5 can be determined according to the size of the particles to be recovered (in this embodiment, the magnetic bead 1). The internal diameter of the tip 5a of the capillary 5 may be, for example, one time or above and two times or less the average particle diameter of the magnetic bead 1.

The height adjustment mechanism 206 is a manipulator that includes, for example, a holding arm 211 and a lifting mechanism 212. The holding arm 211 holds the capillary 5. The lifting mechanism 212 raises and lowers the capillary 5 via the holding arm 211. The lifting mechanism 212 moves the capillary 5 closer to and further away from the array substrate 10. The height adjustment mechanism 206 can hold the capillary 5 at any desired height.

The height adjustment mechanism 206 may also have the function of not only adjusting the height of the capillary 5 but also positioning the capillary 5 at any location within the horizontal plane.

The liquid supply system 207 includes a liquid supply path 213, a pump 214, and a pressure gauge 215. The liquid supply path 213 connects the capillary 5 and the pump 214. The liquid supply path 213 is, for example, a tube made of resin. The liquid supply path 213 is filled with oil (a hydrophobic liquid), for example. The pump 214 can adjust the pressure of the hydrophilic liquid in the capillary 5 by applying pressure to the oil in the liquid supply path 213. The pressure gauge 215 measures the pressure in the liquid supply path 213.

The magnet 208 is composed of a material such as rare-earth magnet material (e.g., neodymium iron boron (NdFeB), samarium cobalt (SmCo)), ceramic magnet material (e.g., strontium ferrite), and other magnetic materials (e.g., iron, cobalt, nickel, their alloys, and oxides). The preferred magnet 208 is a neodymium magnet (NdFeB).

The magnet 208 is installed above the array substrate 10. The magnet 208 is positioned above the housing chamber 11. The magnet 208 can exert a magnetic force (attraction direction magnetic force) on the magnetic bead 1 within the housing chamber 11.

The magnet 208 may have one of its ends, either the upper or lower, as the N pole, and the other end as the S pole. The magnet 208 may also be an electromagnet.

A hydrophobic liquid layer 121 is formed on the inside of the inner peripheral wall 112 of the water-stacking frame 204. The hydrophobic liquid layer 121 is formed on the main surface 10a and encompasses all the housing chambers 11. The hydrophobic liquid layer 121 covers the surface of the first hydrophilic liquid 2 in the housing chambers 11.

Examples of hydrophobic liquids that can constitute the hydrophobic liquid layer 121 include fluorine oil and silicone oil. Examples of fluorine oil include perfluoropolyether (PFPE), perfluoroalkyl ether (PFAE), and perfluoropolyalkyl ether (PFPAE). Other hydrophobic liquids include hydrofluoroether (HFE), mineral oil, saturated hydrocarbon, unsaturated hydrocarbon, aromatic hydrocarbon, and perfluorocarbon.

A hydrophilic liquid layer 122 is formed on top of the hydrophobic liquid layer 121. Examples of hydrophilic liquid that can constitute the hydrophilic liquid layer 122 include water and an aqueous solution. The hydrophilic liquid layer 122 is formed on the inside of the outer peripheral wall 113 of the water-stacking frame 204.

### Magnetic Bead Recovery Method

The bead recovery method according to the embodiment will be described.

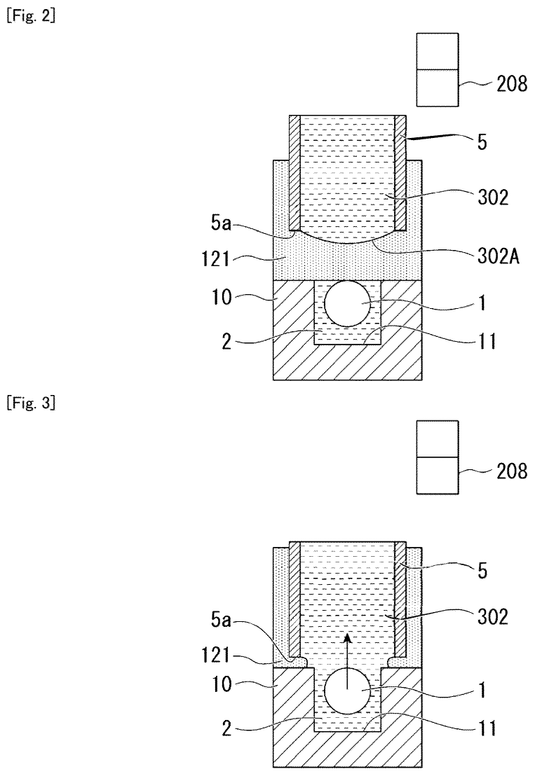

FIGS. 2 to 5 are process diagrams of the magnetic bead recovery method according to the embodiment.

The magnetic bead recovery method of this embodiment includes a first step (fusing the second hydrophilic liquid with the first hydrophilic liquid in the housing chamber), a second step (leading the magnetic bead into the capillary), and a third step (discharging the magnetic bead).

### First Step: Fusing the Second Hydrophilic Liquid With the First Hydrophilic Liquid in the Housing Chamber)

As shown in FIG. 2, the second hydrophilic liquid 302 is introduced into the capillary 5. The second hydrophilic liquid 302 in the capillary 5 reaches the tip 5a. The second hydrophilic liquid 302 protrudes downward from the tip 5a. The second hydrophilic liquid 302 is, for example, water or an aqueous solution. The portion of the second hydrophilic liquid 302 that protrudes from the tip 5a is referred to as “second hydrophilic liquid 302A.” The second hydrophilic liquid 302A has a convex shape, such as a partial spherical or partial ellipsoidal shape.

The magnet 208 applies an upward magnetic force (attractive force) to the magnetic bead 1 within the housing chamber 11. As a result, the magnetic bead 1 in the housing chamber 11 rises within the first hydrophilic liquid 2.

The capillary 5 is positioned to face the housing chamber 11. The capillary 5 is lowered using the height adjustment mechanism 206 (see FIG. 1). The portion of the capillary 5 including the tip 5a is brought closer to the housing chamber 11 through the hydrophobic liquid layer 121. The portion of the capillary 5 including the tip 5a is immersed in the hydrophobic liquid layer 121.

The pressure inside the capillary 5 is preferably adjusted to maintain the state where the second hydrophilic liquid 302A protrudes from the tip 5a. The pressure inside the capillary 5 can be adjusted by the pump 214 (see FIG. 1).

As shown in FIG. 3, when the second hydrophilic liquid 302A (see FIG. 2) reaches the first hydrophilic liquid 2 within the housing chamber 11, the second hydrophilic liquid 302 fuses with the first hydrophilic liquid 2.

### Second Step: Leading the Magnetic Bead Into the Capillary

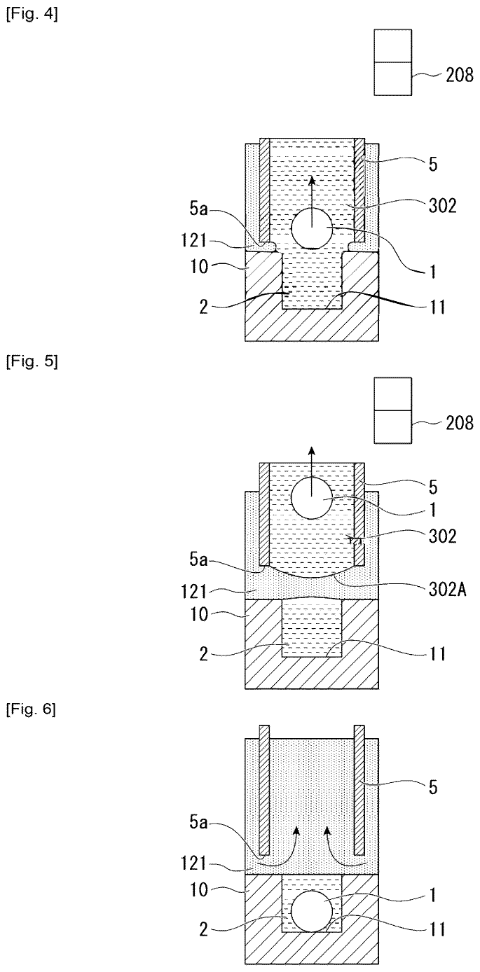

As shown in FIG. 4, due to the magnetic force (attractive force) exerted by the magnet 208, the magnetic bead 1 rises through the first hydrophilic liquid 2 and the second hydrophilic liquid 302 and is guided into the capillary 5.

As shown in FIG. 5, the capillary 5 is raised using the height adjustment mechanism 206 (see FIG. 1). The second hydrophilic liquid 302 in the capillary 5 separates from the first hydrophilic liquid 2 in the housing chamber 11. This allows the recovery of the sample from the housing chamber 11 of the array substrate 10. The recovered sample is either the magnetic bead 1 or a bead suspension (hydrophilic liquid 2 containing the magnetic beads 1).

### Third Step: Discharging the Magnetic Bead

After pulling the capillary 5 out of the hydrophobic liquid layer 121, the magnetic bead 1 is moved to the tip 5a of the capillary 5 by applying the magnetic force (attractive force) of the magnet. By increasing the pressure inside the capillary 5 using the pump 214 (see FIG. 1), the magnetic bead 1 can be discharged from the tip 5a together with a portion of the second hydrophilic liquid 302.

### Effects of the Magnetic Bead Recovery Method of the Embodiment

In the magnetic bead recovery method of this embodiment, the second hydrophilic liquid 302 in the capillary 5 is fused with the first hydrophilic liquid 2 in the housing chamber 11, and the magnetic beads 1 in the housing chamber 11 are guided into the capillary 5 by magnetic force. Therefore, the magnetic beads 1 in the housing chamber 11 can be easily recovered.

In the magnetic bead recovery method of this embodiment, the second hydrophilic liquid 302A is protruded from the tip 5a when the capillary 5 is brought closer to the housing chamber 11. As a result, the second hydrophilic liquid 302 easily fuses with the first hydrophilic liquid 2 in the housing chamber 11.

To clarify the effect of the magnetic bead recovery method of the embodiment, the following comparative magnetic bead recovery methods are shown:

FIG. 6 is an explanatory diagram of the magnetic bead recovery method according to the first comparative embodiment. As shown in FIG. 6, in the magnetic bead recovery method of the first comparative embodiment, there is no hydrophilic liquid in the capillary 5. Therefore, even if the capillary 5 is brought closer to the housing chamber 11 and the inside of the capillary 5 is depressurized, the hydrophobic liquid surrounding the capillary 5 is introduced into the capillary 5, making it difficult to guide the magnetic bead 1 in the housing chamber 11 into the capillary 5.

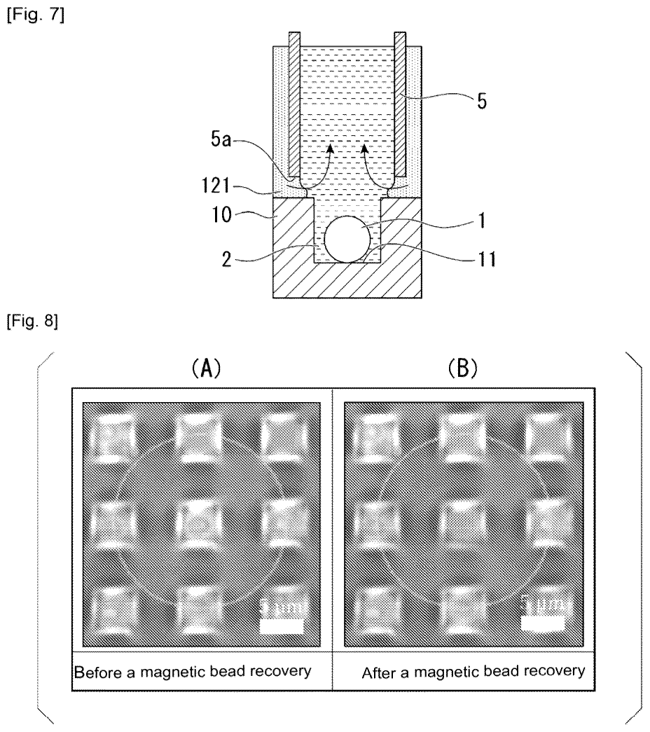

FIG. 7 is an explanatory diagram of the magnetic bead recovery method according to the second comparative embodiment. As shown in FIG. 7, in the magnetic bead recovery method of the second comparative embodiment, no magnet is used. Therefore, even if the second hydrophilic liquid 302 fuses with the first hydrophilic liquid 2, the magnetic beads 1 are not guided into the capillary 5.

### Effects of the Magnetic Bead Recovery Device of the Embodiment

The magnetic bead recovery device 200 of this embodiment includes a height adjustment mechanism 206 that brings the capillary 5 closer to the housing chamber 11, and a magnet 208 that applies an attractive magnetic force to the magnetic bead 1. Therefore, the second hydrophilic liquid 302 in the capillary 5 can be fused with the first hydrophilic liquid 2 in the housing chamber 11, and the magnetic bead 1 in the housing chamber 11 can be guided into the capillary 5 by magnetic force. Thus, the magnetic bead 1 in the housing chamber 11 can be easily recovered.

### EXAMPLES

The present invention will be described based on examples. However, the embodiments of the present invention are not limited to the descriptions of these examples.

### Preparation of Array Substrate with Water Layer Stacked on Oil Layer Encapsulating Bead

### Synthesis of Biotin-Modified DNA

A PCR reaction solution of the following composition was prepared, and PCR was performed for 30 cycles (98 °C for 10 seconds; 55 °C for 5 seconds; 72 °C for 2 minutes). The PCR product was purified using a QIAquick® PCR purification column (QIAGEN) to obtain biotin-modified DNA.

### PCR Reaction Solution

20 pg/μL template DNA
0.3 μM biotin-modified DNA primer
0.3 μM DNA primer
0.2 μM each dNTP mix
0.025 U/μL PrimeSTAR® HS polymerase
1×PrimeSTAR® buffer (Takara Bio)

### Preparation of DNA-Immobilized Magnetic Bead

120 μL of the supernatant of streptavidin-coated magnetic beads (MS 300/streptavidin, JSR; hereinafter referred to as “magnetic beads”) was removed, and the magnetic beads were washed with 100 μL of binding buffer (10 mM Tris-HCl, 1 mM EDTA, 1 M NaCl, 0.05% (w/v) Tween® 20, pH 7.4). The magnetic beads were suspended in 170 μL of binding buffer containing 7 pmol of biotin-modified DNA and agitated at room temperature for 30 minutes, thereby binding the biotin-modified DNA to the magnetic beads. The magnetic beads were then washed five times with 100 μL of binding buffer. Finally, the magnetic beads were suspended in 30 μL of binding buffer.

The concentration of DNA-immobilized magnetic beads in the above suspension was measured using an automatic cell counter (product name: Countess II, Thermo Fisher). The supernatant of the DNA-immobilized magnetic bead suspension (containing 5×10\hat{ }7 DNA-immobilized magnetic beads) was removed, and the DNA-immobilized magnetic beads were suspended in 60 μL of fluorescent aqueous solution (10 μM fluorescein sodium, 50 mM Tris-HCl, pH 7.5). The DNA-immobilized magnetic bead suspension prepared in this manner was used in the following section “Distribution of Beads and Aqueous Solution to the Array Substrate”.

### Fabrication of the Array Substrate

A layer of nickel-phosphorus was plated to a thickness of approximately 100 μm on the surface of a STAVAX® substrate (30 mm×30 mm×5 mm). Using a cutting tool with a blade width of 6 μm, grooves were processed in a grid pattern on the nickel-phosphorus layer, creating a mold with cubic structures (4 μm×4 μm×4 μm) arranged in an area of 1.2 mm×1.2 mm with 1×10\hat{ }6 units. The mold was mounted on an injection molding machine, and cycloolefin polymer (product name: ZEONEX® 480, manufactured by Zeon Corporation) was injected. As a result, an array substrate was fabricated with microwells (4 μm×4 μm×4 um) arranged in an area of 1.2 mm×1.2 mm on the surface of the substrate (30 mm×30 mm×1 mm). Hereinafter, the region of the array substrate where the microwells are arranged is referred to as the “array region,” and the region without microwells is referred to as the “non-array region.”

### Surface Treatment of the Array Substrate

The main surface of the array substrate and the interior surfaces of the microwells were rendered hydrophilic by oxygen plasma irradiation. Subsequently, a polypropylene film vapor-deposited with hexamethyldisilazane (HMDS) was pressed onto the surface of the array substrate to transfer the HMDS to the substrate surface, followed by treatment at 110 °C for 10 minutes to render the substrate surface hydrophobic. Through these operations, an array substrate was fabricated wherein the interior surfaces of the microwells (main surface) are hydrophilic, and the external surfaces of the microwells are hydrophobic.

### Distribution of Bead and Aqueous Solution to the Array Substrate

A wipe member was prepared by inserting a stainless steel rod with a diameter of 3 mm into a silicone tube (outer diameter 4 mm, inner diameter 2 mm, length 50 mm). The wipe member was placed in contact with the non-array region of the array substrate. A neodymium magnet (20×5×10 mm) was placed under the array substrate between the contact part of the wipe member and the edge of the array region.

60 μL of DNA-immobilized magnetic bead suspension was dropped onto the main surface of the array substrate from the contact part of the wipe member to the edge of the array region. 60 μL of fluorine oil (product name: Krytox@ GPL104, manufactured by Chemours) was dropped onto the main surface of the array substrate on the opposite side of the array region from the contact surface of the wipe member.

The wipe member and the neodymium magnet were moved simultaneously toward the array region at a speed of 0.1 mm/s and stopped when they passed the array region. This operation distributed the DNA-immobilized magnetic beads and fluorescent aqueous solution into the microwells and encapsulated them with fluorine oil.

### Stacking of Water on the Oil Layer on the Surface of the Array Substrate

A water stacking frame (liquid stacking frame) was fabricated using a 3D printer (product name: Form 2, Formlabs). The water stacking frame was attached to the array substrate encapsulating DNA-immobilized magnetic beads and fluorescent aqueous solution in the microwells. 800 μL of fluorine oil (product name: Krytox® GPL107, manufactured by Chemours) was added inside the inner peripheral wall of the water stacking frame, forming a hydrophobic liquid layer of fluorine oil on the surface of the array substrate.

After filling the annular groove between the inner and outer peripheral walls of the water stacking frame with 1 ml of water, an additional 3 mL of water was added. This caused the water in the annular groove to overflow, forming a layer of water (hydrophilic liquid layer) on the fluorine oil (hydrophobic liquid layer) inside the inner peripheral wall.

### Recovery of DNA-Immobilized Magnetic Beads from Microwells

The array substrate prepared as described above was placed on the stage of an inverted fluorescence microscope (TI-E, Nikon). A gas-tight syringe (Hamilton) and a pressure gauge (product name: KDM30, KROHNE) were attached to a syringe pump (YMC) and connected to a capillary holder (Eppendorf) via a polyethylene tube (liquid transfer path). This was mounted on a micromanipulator (product name: TransferMan NK2, Eppendorf) installed on the inverted fluorescence microscope (TI-E, Nikon) (see FIG. 1).

The liquid transfer path was filled with mineral oil. The entire glass capillary (tip inner diameter 5.5 μm) was filled with an aqueous solution (50 mM Tris-HCl, pH 7.5) and mounted on the capillary holder. The tip of the glass capillary was pointed downward, and the tip of the glass capillary was observed using coaxial reflected illumination through a half-mirror, adjusting so that the tip of the glass capillary was positioned at the center of the microscope's field of view.

A neodymium magnet (10×10×35 mm) was placed near the glass capillary. By operating the syringe pump, the gauge pressure of the pressure gauge was set to −3 kPa. The manipulator was operated to lower the tip of the glass capillary toward the surface of the array substrate.

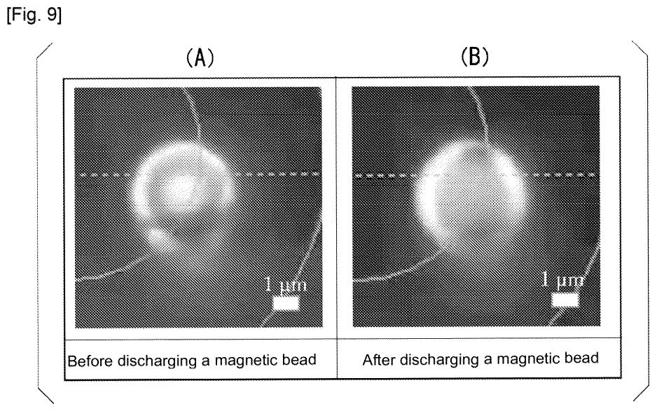

While simultaneously observing the microwell and the capillary tip with coaxial reflected illumination, the glass capillary was lowered directly above the microwell containing the DNA-immobilized magnetic bead. As soon as the aqueous solution at the tip of the glass capillary made contact with the aqueous solution in the microwell, the DNA-immobilized magnetic bead in the microwell rose and moved into the glass capillary. FIG. 8(A) shows a microscopic image of the microwell before magnetic bead recovery. FIG. 8(B) shows a microscopic image of the microwell after magnetic bead recovery. From FIG. 8(A) and 8(B), it can be seen that the magnetic bead was recovered from the microwell.

The glass capillary was raised and removed from the fluorine oil layer. By bringing the glass capillary tip close to the neodymium magnet, the DNA-immobilized magnetic bead was moved to the tip of the glass capillary. The tip of the glass capillary was lowered onto a plastic substrate of the same material and thickness as the array substrate. Observation with coaxial reflected illumination confirmed the presence of DNA-immobilized magnetic bead at the tip of the glass capillary.

By operating the syringe pump, the gauge pressure of the pressure gauge was set to +3 kPa. The tip of the glass capillary was inserted into a droplet of water on the plastic substrate, thereby discharging the DNA-immobilized magnetic bead from the tip of the glass capillary into the droplet.

FIG. 9(A) shows a microscopic image of the magnetic bead and the glass capillary before discharge. FIG. 9(B) shows a microscopic image of the magnetic bead and the glass capillary after discharge. From FIG. 9(A) and 9(B), it can be seen that the magnetic bead was discharged from the glass capillary.

As described above, the embodiments of the present invention have been explained, but the configurations and combinations in the embodiments are merely examples, and additions, omissions, substitutions, and other modifications of the configurations are possible within the scope that does not deviate from the purpose of the present invention.

The number of magnetic bead 1 housed in the housing chamber 11 is not limited to one. The number of magnetic bead 1 housed in the housing chamber 11 can be any number, including multiple (two or more). The number of magnetic bead 1 housed can be adjusted by at least one of the inner dimension and depth of the housing chamber 11. If the number of magnetic bead 1 housed in the housing chamber 11 is multiple, the number of bead to be analyzed per unit area of the array substrate 10 can be increased. Thus, screening can be performed efficiently.

The contact angle of water on the hydrophobic surface is, for example, 60 degrees or more. The contact angle of water on the hydrophilic surface is, for example, 40 degrees or less.

The magnetic bead may have a nucleic acid and a protein immobilized on it. After evaluating the function of the protein within the housing chamber, the magnetic bead can be recovered using the recovery method described in the embodiment. By analyzing the nucleic acid immobilized on the recovered magnetic bead, the amino acid sequence of the protein can be identified (see International Publication No. 2020/095985, for example).

The magnetic bead may have a nucleic acid immobilized on it. After evaluating the function of the nucleic acid within the housing chamber, the magnetic bead can be recovered using the recovery method described in the embodiment. By analyzing the nucleic acid immobilized on the recovered the magnetic bead, the sequence of the nucleic acid can be identified (see, for example, Aniela Wochner et al., Ribozyme-Catalyzed Transcription of an Active Ribozyme. Science 332, 209 (2011)).

The magnetic bead may have a nucleic acid immobilized on it. After lysing the cell within the housing chamber and hybridizing an intracellular RNA to the nucleic acid on the magnetic bead, the magnetic bead can be recovered using the recovery method described in the embodiment. The RNA hybridized to the nucleic acid immobilized on the recovered magnetic bead can be analyzed (see Jinzhou Yuan et al., An Automated Microwell Platform for Large-Scale Single Cell RNA-Seq. Sci. Rep. 6:33883(2016 ), for example).

The magnetic bead may have an antibodie immobilized on it. After lysing the cell within the housing chamber and binding a cell-derived protein to the antibody on the magnetic bead, the magnetic bead can be recovered using the recovery method described in the embodiment. The proteins bound to the antibodie immobilized on the recovered magnetic bead can be analyzed (see Lucas Armbrecht et al., Single-cell protein profiling in microchambers with barcoded beads. Microsystems & Nanoengineering 5:55 (2019), for example).

### REFERENCE SIGNS LIST

1: Magnetic bead
2: First hydrophilic liquid
5: Capillary
5a: Tip
10: Array substrate (substrate)
10a: Main surface (one surface)
11: Housing chamber
121: Hydrophobic liquid layer
206: Height adjustment mechanism (movement mechanism)
208: Magnet
302: Second hydrophilic liquid

1. A method for recovering a magnetic bead from a housing chamber containing the magnetic bead and a first hydrophilic liquid, covered by a hydrophobic liquid layer, comprising:
a step of bringing a capillary, filled with a second hydrophilic liquid up to its tip, close to the housing chamber through the hydrophobic liquid layer, and merging the second hydrophilic liquid with the first hydrophilic liquid in the housing chamber; and
a step of guiding the magnetic bead into the capillary through the first and second hydrophilic liquids by magnetic force.

2. The method for recovering the magnetic bead according to claim 1, wherein the second hydrophilic liquid is protruded from the tip when bringing the capillary close to the housing chamber.

3. The method for recovering the magnetic bead according to claim 1, wherein the housing chamber is a recess formed on one surface of the substrate.

4. The method for recovering the magnetic bead according to claim 1, further comprising a step of discharging the magnetic bead from the capillary.

5. A device for a magnetic bead recovery comprising:
a capillary positioned opposite a housing chamber containing a magnetic bead and a first hydrophilic liquid;
a magnet applying a magnetic attraction force to the magnetic bead;
a movement mechanism for moving the capillary toward and away from the housing chamber;
wherein a hydrophobic liquid layer covering the first hydrophilic liquid is formed in the housing chamber; and
the movement mechanism brings the capillary, filled with a second hydrophilic liquid up to its tip, close to the housing chamber through the hydrophobic liquid layer.
